(19) United States
(12) Patent Application Publication
(10) Pub. No.: US 2015/0335297 A1
(43) Pub. Date: Nov. 26, 2015

(54) Movable radiologically protective enclosure for a physician or medical technician

(71) Applicants: Jamil MOGUL, Saratoga, CA (US); Anatoly RYZHIKH, Moscow (RU)

(72) Inventors: Jamil MOGUL, Saratoga, CA (US); Anatoly RYZHIKH, Moscow (RU)

(21) Appl. No.: 14/703,880

(22) Filed: May 4, 2015

Related U.S. Application Data
(60) Provisional application No. 61/987,859, filed on May 2, 2014.

(51) Int. Cl. A61B 6/10

(52) U.S. Cl. CPC A61B 6/107

(57) ABSTRACT

A movable and radiologically protective enclosure for a physician or medical technician working in close proximity to a source of radiologic energy. The embodiments include a lower assembly for enclosing and radiologically shielding the lower torso and legs of the user, and for supporting an upper assembly designed to enclose and radiologically shield the upper torso and arms of the user. A transparent radiologically impervious dome or the like is carried by the upper assembly to receive and protect the head of the user as well as allow visual and functional access to patients and/or radiologic equipment Means are provided for allowing the upper assembly to be vertically adjusted in position and to angularly tilt the torso and/or head protecting components. Protective sleeves are also provided at frontal ports for allowing the user to protectedly reach into the work area.

### RELATED APPLICATION

This application claims priority to related U.S. Provisional Application No. 61/987,859, bearing the same title and filed May 2, 2014. The application in its entirety is expressly incorporated herein by reference.

### BACKGROUND

1. Field of the Invention

The present invention relates generally to radio-protective apparatus and more particularly to an x-ray protection cabin for use by medical professionals and others likely to be exposed to ionizing radiation of the X-ray type and the like.

2. Prior Art

As disclosed in the Magram U.S. Pat. No. 5,185,778, modern operating rooms often include a table on which a patient lies during an operating procedure, and diagnostic equipment in the form of an x-ray source and an x-ray detector, is located on a gantry with the patient and operating table between the x-ray source and detector. The gantry is rotatable about a horizontal axis so that a surgeon viewing a display responsive to the detector is provided with real time display of the tissue being operated on. During the procedure, the patient is irradiated by x-rays for prolonged intervals and is either moved relative to the source of x-rays by providing the table with motors and a linkage that move the table and patient relative to the platform, or held in a fixed position by holding the table in situ relative to the platform.

During the operation the surgeon and one or more assistants standing next to the operating table are constantly exposed to x-rays back-scattered from the patient and/or table unless adequate shielding is provided. It is well known that the accumulated effect of the back-scattered radiation over many years of conducting surgical procedures may have deleterious effects on the health of the surgeon and assistant(s), and may induce cancer.

To reduce the x-ray exposure to the surgeon and assistant(s) while the x-ray source is irradiating a patient, they usually wear leaded eyeglasses, a lead thyroid covering and a lead apron which covers the chest, abdomen and thighs but leaves uncovered the arms, hands, legs below the knees and head. However it is known that such radiation protection is only partially effective in blocking radiation and leaves substantial parts of the body uncovered, and that amount of back-scattered x-ray radiation incident on the surgeon and assistant(s) can be substantial enough to cause radiation damage to the surgeon and assistant(s) when they are exposed over a prolonged time period.

In some instances lead shield panels that are transparent to optical energy but substantially opaque to x-rays are provided for the surgeon and assistant(s) to stand behind. Such shield panels are typically unitary structures fixedly mounted on caster supported frames to allow a degree of freedom of movement. The panels are usually made of lead glass having sufficient thickness to substantially attenuate the back-scattered x-rays and thereby provide at least some protection for the surgeon and assistants. However the panels often do not enable the surgeon and/or assistants easy access to the patient during the operating procedure. Consequently, if access to the patient is necessary during the procedure and while the patient is being x-rayed, there is a substantial likelihood that sensitive body portions of the surgeon and/or assistant(s) will be irradiated with undesirable doses of back-scattered x-ray radiation.

In a subsequently issued U.S. Pat. No. 7,465,947, Magram discloses a shielding apparatus wherein back-scattered x-rays incident on a surgeon or assistant working with a patient on an operating table can be reduced by upper and lower x-ray shield panels mounted so they can be turned relative to each other about a common vertical axis. The panels are thick enough and have proximate horizontal edges close enough to each other to substantially attenuate back-scattered x-rays incident on front faces of the panels when the panels are aligned. The panels are individually pivotal about a vertical axis to form an opening enabling the user's hand(s) and forearm(s) to extend through. A hinge connects the upper and lower panels to a third panel. In a second embodiment, the upper and lower panels are moved up and down together, while the lower panel remains aligned with a fixed fourth panel for protecting the user's legs.

In U.S. Pat. No. 7,112,811, Lemer discloses a caster mounted protective screen assembly including a patient facing front wall fixedly connected to a lateral wall running perpendicular, or essentially perpendicular, to the front wall. The walls include transparent panels extending over at least a part of the height thereof. The upper part of the front wall is inclined slightly forwards, forming an overhang which permits the user to approach the working region, and further includes two holes for permitting passage of the user's arms into the working region. In an embodiment, a protective drape or curtain is pivotally connected to the rearmost extremity of the lateral wall to provide a degree of shielding of the user's back. However, no protection is provided on the fourth side of the apparatus.

While such attempts to address the problems associated with the exposure of surgeons and assistants to radiation during radiological surgery and diagnosis, there remains a need for an improved x-ray screening apparatus which is more convenient to use, which offers better visibility of the surgeon's work region, and which provides better protection of surgeons and assistants from exposure to scattered x-ray radiation.

Accordingly, it is an object of the present invention to provide a new and improved method of and apparatus for shielding surgeons and/or assistants from scattered x-ray radiation during surgery while x-rays are being used for diagnostic purposes.

Another object of the present invention is to provide a new and improved apparatus for shielding critical body parts of surgeons and/or assistants during an operating procedure that is accompanied by patient exposure to diagnostic x-ray radiation.

Another object of the present invention is to provide a new and improved shielding apparatus having features which enable relatively easy access to the patient.

Still another object of the present invention is to provide on the one hand better visibility of the user's work region, and on the other hand greater comfort while positioned behind the screen within the framework of his intervention.

Yet another object is to provide better working conditions for surgeons, medical technitions and assistants during the conduct of radiologic procedures, without requiring the wearing of heavy protective clothing, and which thus enables conduct of the intervention with greater accuracy, higher efficiency, and a greater degree of safety.

A further object of the present invention is to provide a new and improved shielding apparatus for use in an operating room environment including x-ray diagnostic equipment, wherein the shielding arrangement includes a movable that is relatively easily moved, despite the substantial weight and density of its shielding components.

A still further object of the present invention is to provide a new and improved shielding apparatus for use in an operating room environment including x-ray diagnostic equipment, wherein the leaded rubber used for radiation protection is encased in a fabric so that the user has no direct contact with the lead; an added safety feature.

### SUMMARY OF THE INVENTION

Briefly, a presently preferred embodiment of the present invention provides a movable radiologically protective enclosure for a physician, assistant or medical technician working in close proximity to a source of radiologic energy. The embodiment includes a lower assembly for enclosing and shielding the lower torso and legs of the user, and for adjustably supporting an upper assembly designed to enclose and shield the upper torso, head and arms of the user as well as allow improved visual and functional access to patient and/or radiologic equipment. To counter balance the weight of the upper assembly, a suitable gas spring, or the like, may be used.

The lower assembly includes a wheeled lower structural framework, preferably made of rectangular tube stock or the like, for supporting frontal and partial lateral radiologic shield components as well as a pair of pivotally mounted radiologic shielding and enclosing components associated with opposite sides of the frontal and lateral shield components. The enclosure components are designed to be rotatable between open and at least substantially closed positions to allow user ingress and egress to and from the enclosure.

The upper assembly also includes a structural framework similarly adapted to carry radiologic shielding including flexible, frontal and partial lateral radiologic shielding components including protective draping material, or the like, for shielding the upper torso of the user. The upper shielding is provided with frontal portals that are sleeved with flexible protective material to allow the user to protectively extend his or her hands and arms through the sleeves and into the patient region. The sleeves are detachable and re-attachable via snap buttons, Velcro fasteners, or any other suitable means. As in the lower assembly, the rearmost sides of the upper assembly are pivotally connected to open and at least substantially close as required for user ingress and egress.

The upper structural framework is also adapted to support a radiologically protective, at least partially transparent dome or cap component, preferably made of lead glass or clear lead plastic material, into which the user's head will extend to provide a full range of visibility. In one embodiment, this component is pivotally attached to the frontal portion of the supporting upper framework and is linked to the supporting frame with adjustable supports so that it can be selectively tilted forward to accommodate the user's visual needs and comfort. To counter balance the weight of the cap or dome, a suitable gas spring, or the like, may be used.

Advantageously, an X-ray radiation impervious skirt is hung from the lower perimeter of the dome or cap to block any radiation that might have a tendency to pass under the back portion of the dome or cap when it is in the tilted forward disposition. Moreover, all of the drapes, skirt pieces, strips and shielding linings are preferably made of fabric-encased lead impregnated rubber material or the like.

The upper structural framework of the upper assembly is advantageously connected to the lower structural framework in a manner so as to allow vertical relative movement therebetween to accommodate different user heights as well as to allow retraction of the upper unit into the lower assembly for storage space conservation when the apparatus is not in use. The relative movement may be advantageously facilitated by the use of frictionless slides, telescoping tubular members or other suitable means. In addition, gas springs, or functionally similar counter-balancing means, may be used to assist in the raising and lowering of the upper assembly relative to the lower assembly.

Also, to facilitate the selective positioning of the upper assembly relative to the lower assembly, a suitable jack or screw mechanism, or the like, may be advantageously used to lift and lower the upper assembly to an intended height position relative to the lower assembly, and a pawl and ratchet mechanism, or the like, may be used to lock the upper assembly in place.

### BRIEF DESCRIPTION OF THE SEVERAL FIGURES OF THE DRAWING

FIG. 1 is a side elevational view of an embodiment of the present invention;

FIG. 2 is a front elevational view of the embodiment of FIG. 1;

FIG. 3 is a rear elevational view of the embodiment of FIG. 1;

FIG. 4 is a side elevational view showing structural details of the lower sub-assembly of the embodiment of FIG. 1;

FIG. 5 is a front elevational view showing structural details of the lower assembly of the embodiment of FIG. 1;

FIG. 6 is a rear elevational view showing structural details of the lower assembly of the embodiment of FIG. 1;

FIG. 7 is a cross sectional view taken along the line 7-7 in FIG. 4;

FIG. 8 is an enlarged view of the portion of FIG. 7 indicated by the arrow 8;

FIG. 9 is a side elevational view showing structural details of the lower structural assembly of the embodiment of FIG. 1;

FIG. 10 is a rear elevational view showing structural details of the upper structural assembly of the embodiment of FIG. 1;

FIG. 11 is a cross sectional view taken along the line 11-11 in FIG. 9;

FIG. 12 is a cross section taken through the base ring of the dome unit depicted in the embodiment of FIG. 1;

FIG. 13 is an enlarged view of the portion of FIG. 12 indicated by the arrow 13 in FIG. 12;

FIG. 14 is a side elevational view showing structural details of the mating of the upper structural assembly of FIG. 10 to the lower structural assembly of FIG. 9;

FIG. 15 is a rear elevational view showing structural details of the mated assembly of FIG. 14;

FIGS. 16 and 17 are respectively side and rear elevations showing the fully assembled embodiment of FIG. 1 with the dome tilted forward, all radialogically protective elements installed on the surer-structure of the apparatus, and the upper assembly lowered to its lowermost position;

FIG. 18 is a stylized partial top down view taken in the plane suggested by the line 18-18 of FIG. 14;

FIG. 19 is a cross section taken along the line 19-19 of FIG. 16;

FIG. 20 is an enlarged diagram showing details of the area 20 in FIG. 19;

FIG. 21 is a side elevational view showing the structural assembly of an alternative embodiment of the present invention;

FIG. 22 is a side elevational view showing structural details of the lower sub-assembly of the embodiment of FIG. 21;

FIG. 23 is a top plan view of the lower sub-assembly shown in FIG. 22;

FIG. 24 is a front elevational view of the lower sub-assembly shown in FIG. 22;

FIG. 25 is a side elevational view showing structural details of the upper sub-assembly of the embodiment of FIG. 21;

FIG. 26 is a top plan view of the upper sub-assembly shown in FIG. 26;

FIG. 27 is a front elevational view of the upper sub-assembly shown in FIGS. 25 and 26;

FIG. 28 is a side elevational view showing structural details of the attachment of the curved hinged bar included in the upper sub-assembly of the embodiment of FIG. 21;

FIG. 29 is a top plan view of the curved horizontal member of the upperframe sub-assembly shown in FIG. 26 showing structural details of the attachment of the curved hinged side bars included in the upper frame sub-assembly of the embodiment of FIG. 21;

FIG. 30 is a top plan view showing the trapezoidally configured cube shaped cap attached to the C-shaped upper structure of the embodiment shown in FIG. 21; and

FIG. 31 is an elevational view showing the trapezoidally configured cube shaped cap attached to the C-shaped upper structure of the embodiment shown in FIG. 21.

### DETAILED DESCRIPTION OF THE EMBODIMENTS

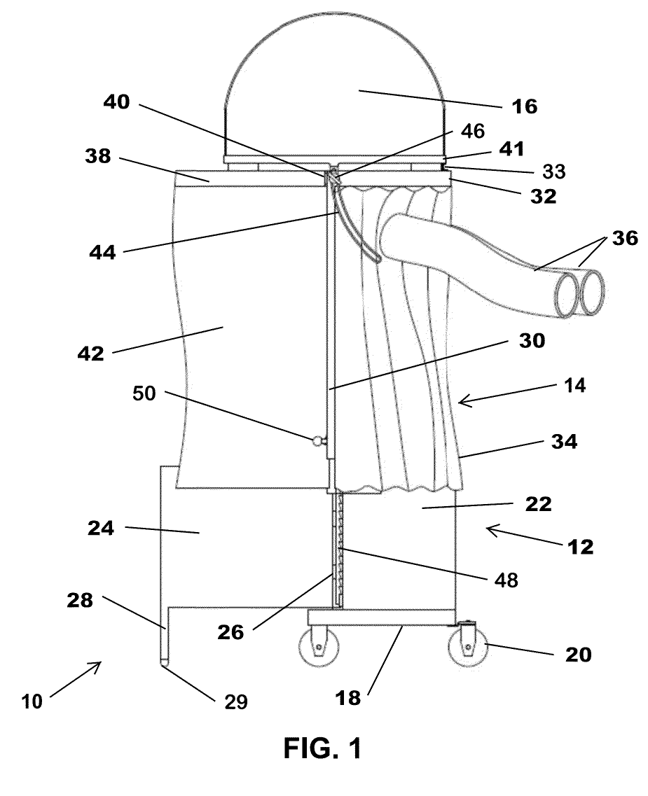

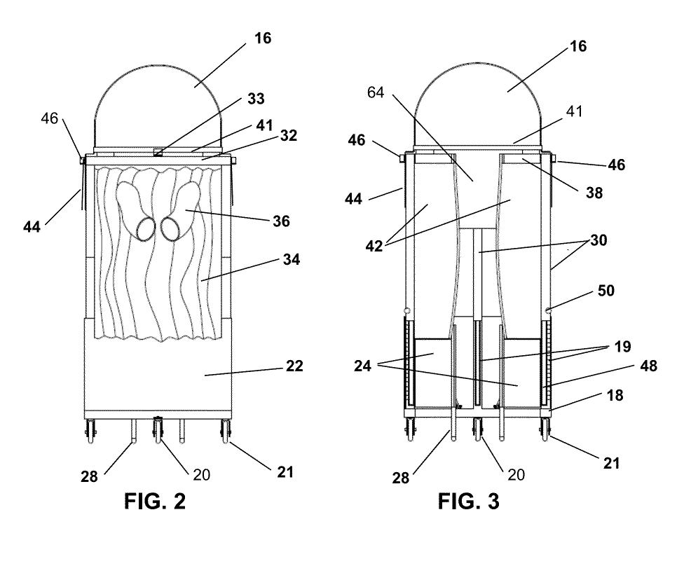

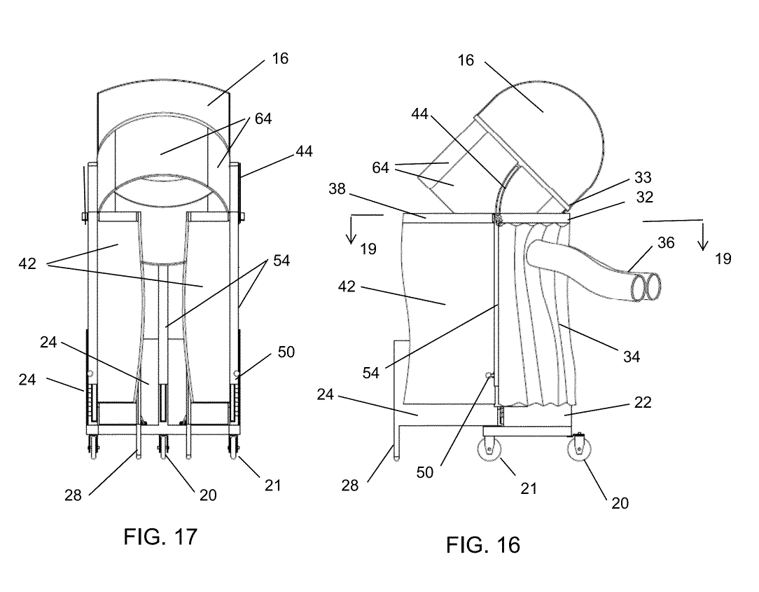

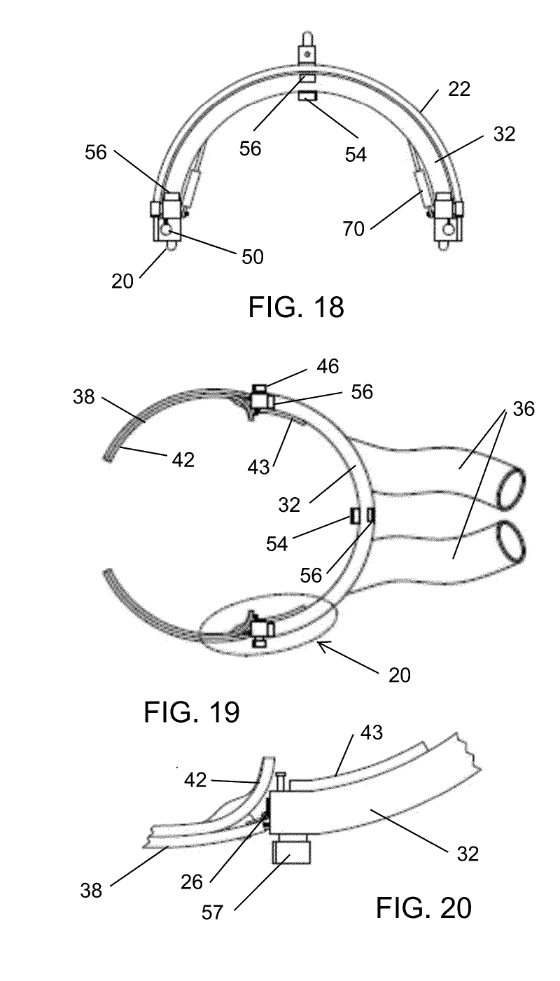

Referring now to FIGS. 1-3 of the drawing, an embodiment of the present invention, generally referred to as an X-shield cabin, is shown at 10 and includes a lower sub-assembly unit 12 for enclosing and shielding the lower torso and legs of a user, and for supporting an upper sub-assembly unit 14 designed to envelope and shield the upper torso and arms of the user as well as allow functional access to patients and/or radiologic equipment. A transparent dome 16, or the like, made of leaded glass or clear lead plastic material is attached to a C-shaped upper horizontal frame member 32 of the upper sub-assembly 14 by a front hinge 33. For the convenience of the user, the dome 16 may be selectively tilted forward (as depicted in FIG. 16) about hinge 33 and held in place by slotted curved support links 44 affixed to the base ring or rim 41 of the dome assembly and matching locking thumb screws 44 affixed to frame member 32 and disposed on opposite sides of the dome.

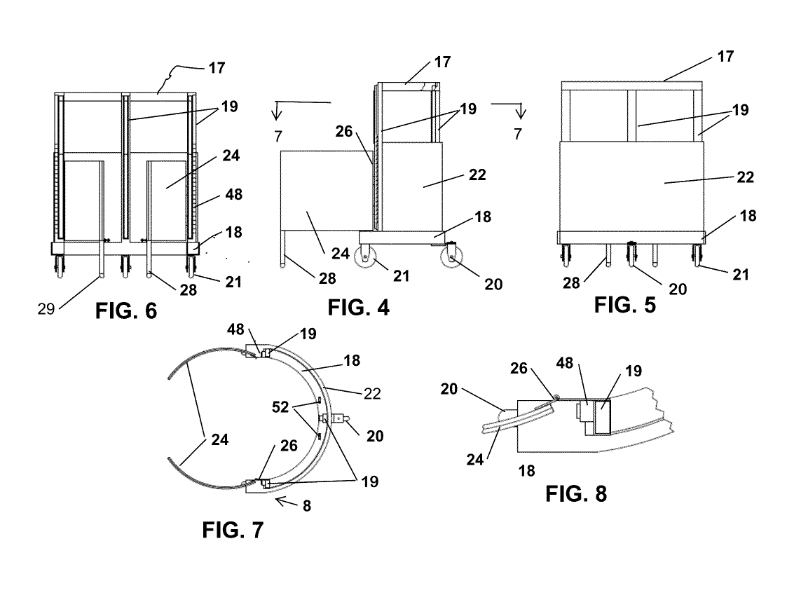

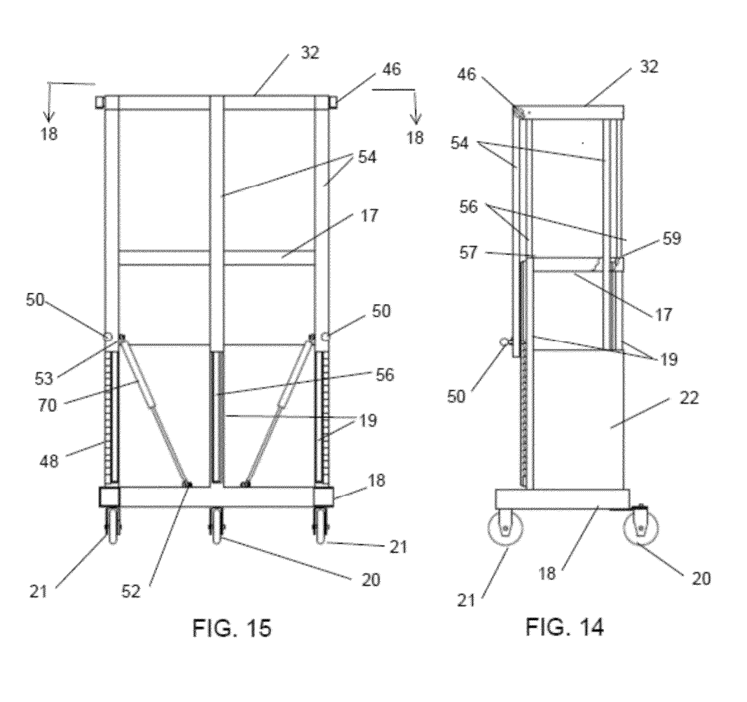

The structural components of the lower sub-assembly unit 12 are best depicted in FIGS. 4-8 and include a lower supporting framework provided by a C-shaped horizontal base member 18 and three hollow, vertically extending column members 19 welded thereto. As will be illustrated below in FIGS. 14 and 15, the hollow columns 19 telescopically receive corresponding vertical guide rods 56 of the upper frame sub-assembly and allow the upper unit to be selectively raised and lowered relative to the lower unit to accommodate different user height requirements. The lower portions of the side columns 19 also have ratchets 26 affixed to their rearwardly facing sides. The lower framework is advantageously mounted on three wheels including a front caster 20 and two fixed position side wheels 21 affixed to the C-shaped horizontal frame member 18 to allow mobility of the cabin within the hospital environs.

The lower framework is also preferably made of rectangular tube stock and supports a frontal and partial lateral radiologic shield component 22 as well as a pair of rearwardly disposed radiologic shield and enclosure components 24 respectively associated with each side of the frontal and lateral shield component 22. These lower front and rear enclosure components are preferrably rigid and covered with a lead impregnated rubber lining.

Each rearwardly extending component 24 is hingedly connected by one or more hinges 26 to a vertical column member 19 (FIGS. 3, 4 and 8), and are designed to be rotatable between open and at least substantially closed positions to partially envelope the back side of the user when in use, and yet allow convenient ingress and egress to and from the cabin. The rearmost extremities of the panels 24 are at least partially supported by legs 28, the lower ends of which have rubber or soft plastic pads 29 affixed thereto to serve as frictional stops tending to resist unwanted movement of the cabin.

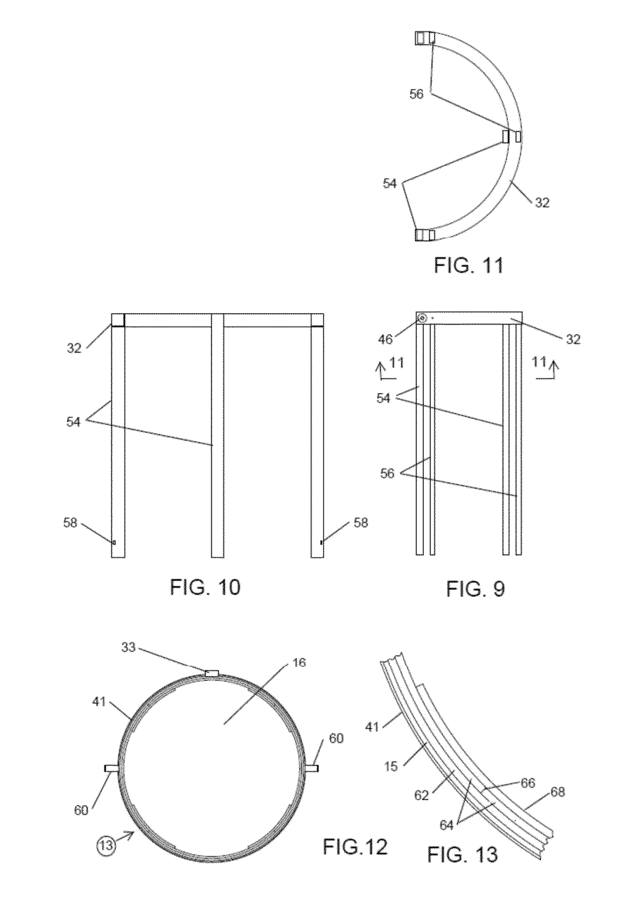

As best shown in FIGS. 9-11, the upper unit and sub-assembly 14 includes an upper structural framework comprised of the C-shaped horizontal member 32 (FIG. 11) and three sets of vertically depending columns and guide members preferably welded to the underside of member 32. The columns 54 overlap the rachets, provide rigidity for the sub-assembly and carry the spring loaded pawls 50 (not shown, but see FIG. 1) attached at apertures 58 provided proximate the lower extremities of the columns. The guide rods 56 telescopically mate with the columns 19 of the lower sub-assembly as described above. The pawls engage the teeth of the ratchets to allow selective positioning of the upper sub-assembly.

The upper sub-assembly provides support for the dome 16 and may include a curved plastic panel wrapped about the upper frontal port of the frame. In such case, two or more apertures would typically be provided therein defining portals though which the user may extend his arms. As illustrated in several figures of the drawing, the upper framework is adapted to carry radiologic shielding including flexible, protective draping material, or the like, both inside and outside the plastic panel for shielding the upper torso of the user. The frontal portion of any upper shielding is also provided with frontal portals that are aligned with the hardware portals and are sleeved with flexible protective material to allow the user to protectively extend his or her hands and arms through the frontal portion of the protective shielding and into the work region. The sleeves are usually detachable and re-attachable via snap buttons, Velcro or any other suitable means. The exterior draping material has holes through which the sleeves extend. As in the lower assembly, the rearmost sides of the upper assembly are pivotally connected to open and at least substantially close as required for user ingress and egress.

Referring specifically to FIGS. 14 and 15, illustrations are provided showing how the upper structural framework of the upper unit is advantageously connected to the lower structural framework by the telescopic entry of the guide rods 56 into the upper ends of the open columns 19 at 57 and 59 in a manner so as to conveniently allow vertical relative movement there between to accommodate different user heights as well as to allow retraction of at least a portion of the upper unit into the lower unit for storage space conservation when the apparatus is not in use. The relative movement may be advantageously facilitated by the use of frictionless slides, telescoping tubular members or other suitable means. In addition, gas springs 70, or functionally similar counter-balancing means, may be used to assist in the raising and lowering of the upper assembly relative to the lower assembly. Also, to facilitate the selective positioning of the upper assembly relative to the lower assembly, a suitable jack or screw mechanism, or the like, may be used to lift and lower the upper assembly to an intended height position relative to the lower assembly, and as illustrated, the pawl and ratchet mechanism, or the like, may be used to lock the assembly in place once it is adjusted.

As mentioned above and further illustrated below in FIGS. 16 and 17, the upper structural framework is also adapted to support the radiologically protective, at least partially transparent dome or cap component 16, preferably made of lead glass or clear lead plastic material, into which the user's head will extend. In the present embodiment, this component is pivotally attached to the frontal portion of the supporting upper framework and linked to the supporting frame with adjustable supports so that it can be tilted forward to accommodate the user's visual comfort. To counter balance the weight of the cap or dome, a suitable gas spring, or the like, may be used.

Advantageously, and as illustrated in FIGS. 12 and 13, as well as in FIGS. 16 and 17, an X-ray radiation impervious skirt comprised of multiple segments 64 of suitable material is hung from the lower perimeter of the dome or cap to block any radiation that might have a tendency to pass under the back portion of the dome or cap when it is in the tilted forward disposition. In the illustrations of FIGS. 12 and 13, it will be noted that the upper edges of the radiation impervious material are suitably affixed to an interior dome edge ring 62 disposed inside the dome edge and in alignment with the dome edge circumscribing ring 41. Note also that all of the material junctions, for example, at the hinged junctions of the rear drape supporting arms 38 to the ends of the C-shaped frame member 32, as shown in FIGS. 19 and 20, are also overlapped by other pieces of the drape material to insure that no scattered radiation is allowed to leak through. All of the drapes, skirt pieces, strips and shielding linings are preferably made of fabric-encased lead impregnated rubber material or the like.

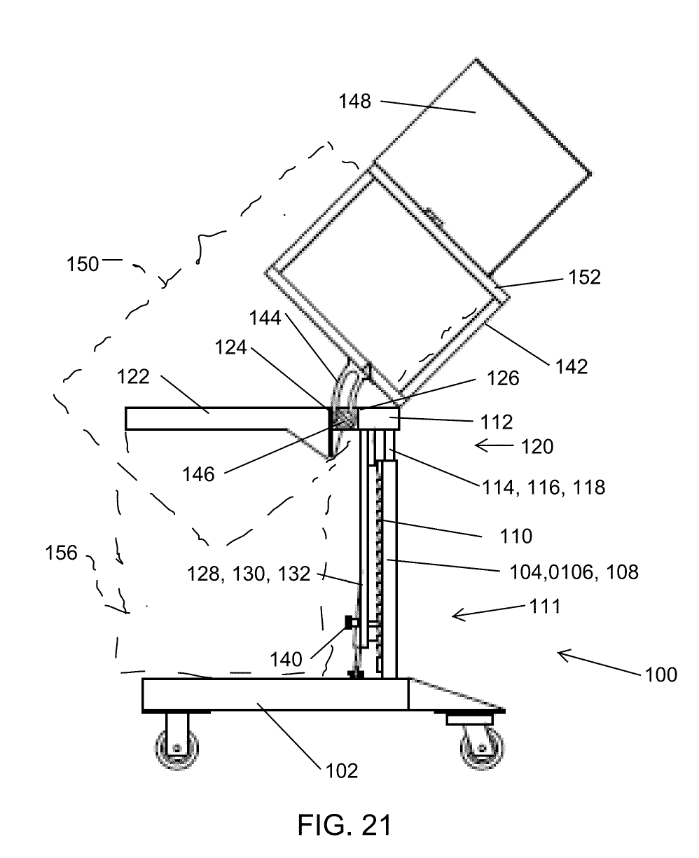

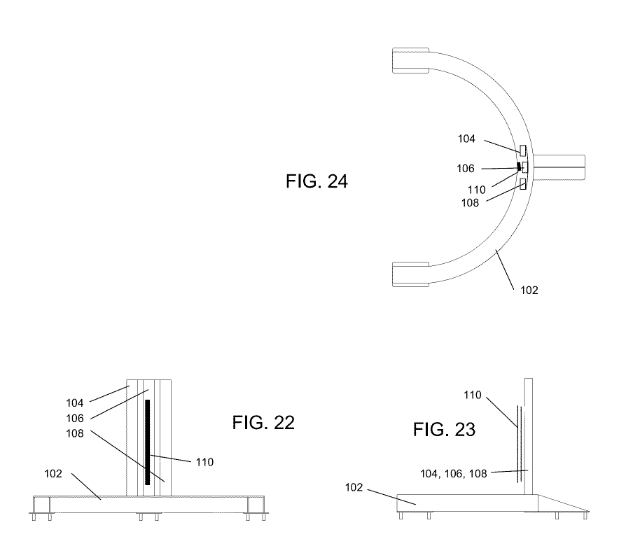

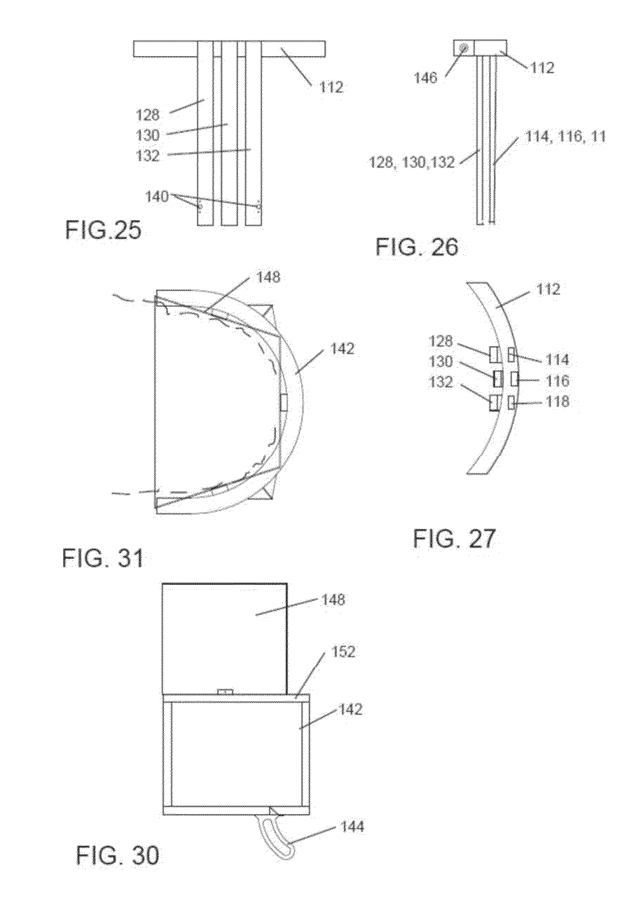

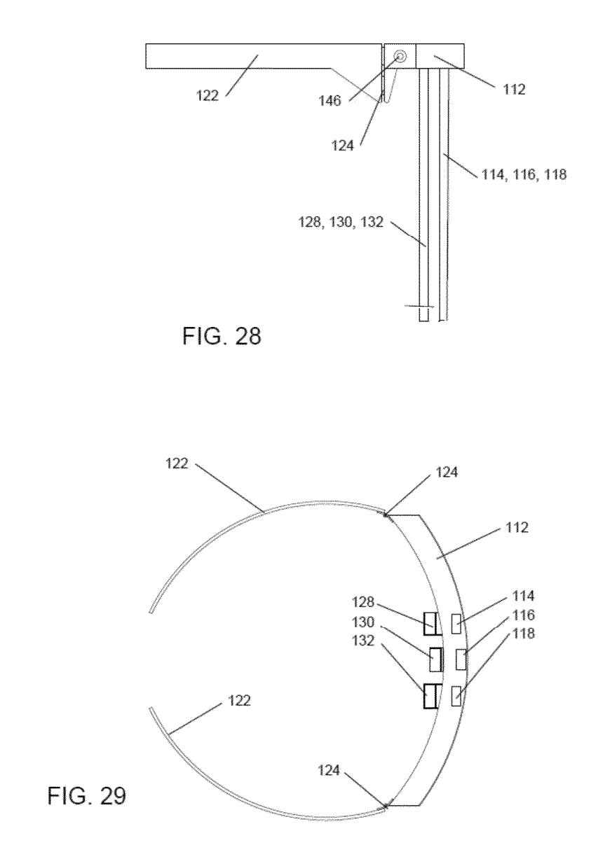

In FIGS. 21-31 details of the underlying superstructure of an alternative embodiment of the present invention are illustrated at 100. In this embodiment, a generally C-shaped horizontal base member 102 is combined with three centrally located (relative to the center part of the :“C”), parallel and vertically extending hollow column members 104, 106 and 108 (see FIGS. 23 and 24 in particular) to provide a base frame assembly 111. The hollow column members form slides, and a ratchet 110 is affixed to the rear side of the center column 106.

A corresponding but truncated C-shaped upper horizontal member 112 is combined with three vertically extending parallel rods 114, 116 and 118 that are respectively adapted to telescopically mate with the three column members 104, 106 and 108, and form a top frame assembly 120 that in addition includes a pair of rearwardly extending curved drape support bars 122 which are hingedly attached at 124 to the truncated arms 126 of the top horizontal member 112. In addition, three vertical frame members 128, 130 and 132 extend downwardly from member 112 in spaced apart parallel relationship to the rods 114, 116 and 118. A spring loaded pawl 140 is carried by the centermost vertical frame member 130 proximate its lower end to engage the ratchet 110.

Lying above and hingedly attached at to the upper member 112 is a generally C-shaped partial upper enclosure structure 142 that is selectively secured in an upwardly rotated position relative to the horizontal plane of top frame member 112 by a slotted bracket 144 and thumb screw 146.

Secured to the top of and rotatable therewith is a transparent cap 148 for receiving the head of a user. Not shown are a generally C-shaped upper drape open at the back and represented by the dashed lines 150. This radilogically protective drape is suspended from the top rim 152 of the upper enclosure structure 142. A second lower drape, open at the back and represented by the dashed lines 156 is suspended from the support arms 122 of the upper framework structure.

1. A movable and radiologically protective enclosure for a physician or medical technician working in close proximity to a source of radiologic energy, comprising:
a lower assembly for enclosing and radiologically shielding the lower torso and legs of the user,
an upper assembly for enclosing and radiologically shielding the upper torso and arms of the user;
a transparent radiologically impervious dome or the like carried by the upper assembly to receive and protect the head of the user as well as allow visual access to patients and/or radiologic equipment ; and
adjustment means for allowing the upper assembly to be vertically adjusted in position and to angularly tilt the torso and/or head protecting components.

2. A movable and radiologically protective enclosure for a physician or medical technician working in close proximity to a source of radiologic energy as recited in claim 1 and further comprising comprising:
at least one frontal port having an associated radiologically protective sleeve for allowing the user to protectedly reach into an irradiated work area.

3. A movable and radiologically protective enclosure for a physician or medical technician working in close proximity to a source of radiologic energy as recited in claim 1 wherein said lower assembly includes means supporting the protective enclosure above a floor upon which it rests and allowing it to be conveniently moved from place to place by the user.
